(19) United States
(12) Patent Application Publication
(10) Pub. No.: US 2015/0216417 A1
(43) Pub. Date: Aug. 6, 2015

(54) DIAGNOSTIC INSTRUMENT AND METHODS RELATING TO RAMAN SPECTROSCOPY

(71) Applicant: NATIONAL UNIVERSITY OF SINGAPORE, Singapore (SG)

(72) Inventors: Zhiwei Huang, Singapore (SG); Mads Sylvest Bergholt, Singapore (SG); Wei Zheng, Singapore (SG); Khek Yu Ho, Singapore (SG)

(21) Appl. No.: 14/421,979

(22) PCT Filed: Aug. 16, 2013
(86) PCT No.: PCT/SG2013/000351

§ 371 Date: Feb. 16, 2015

Related U.S. Application Data
(60) Provisional application No. 61/683,761, filed on Aug. 16, 2012.

(51) Int. Cl. A61B 5/00

(52) U.S. Cl. CPC A61B 5/0075; A61B 5/441; A61B 5/7221; A61B 5/7282

(57) ABSTRACT

A probe head for a diagnostic instrument, the probe head comprising;
a transmission optical fiber,
a plurality of collection optical fibers, and
a lens to transmit light from the transmission optical fiber to a test site,
wherein the ends of the collection optical fibers are bevelled.

### BACKGROUND TO THE INVENTION

Raman spectroscopy is a technique which uses inelastic or Raman scattering of monochromatic light. Conventionally, the monochromatic light source is a laser in the visible or near infrared (“NIR”) range. The energy of the scattered photons is shifted up or down in response to interaction with vibrational modes or excitations in the illuminated material, varying the wavelength of the scattered photons. Accordingly, the spectra from the scattered light can provide information about the scattering material.

It is known to use NIR Raman spectroscopy as a potential technique for characterisation and diagnosis of precancerous and cancerous cells and tissue in vivo in a number of organs. The technique is desirable as it can be non-invasive or minimally invasive, not requiring biopsies or the other removal of tissue. It is known to use NIR Raman spectroscopy in two wavelength ranges. The first is the so-called fingerprint (“FP”) range, with wave numbers from 800 to 1800 cm⁻¹, owing to the wealth of highly specific bimolecular information, for example from protein, DNA and lipid contents, contained in this spectral region for tissue characterisation and diagnosis. The disadvantage of this wavelength range is, that when used with a commonly used 785 nm laser source, the illuminated tissue autofluoresces, generating a strong background (‘AF’) signal. Further, where the probe uses optical fiber links, a Raman signal is scattered from the fused silica in the optical fibers. In particular, where a charge-coupled device (“CCD”) is used to measure the scattered spectra, the autofluorescence signal can saturate the CCD and interfere with the detection of the comparatively weak Raman signals in this wavelength area.

It is also known to measure Raman scattering in a relatively high wavenumber range (“HW”) with wavenumbers in the range 2800 to 3700 cm⁻¹. This wavenumber range is desirable as strong Raman signals are generated from CH₂ and CH₃ moiety stretching vibrations in proteins and lipids, and OH stretching vibrations of water, desirable for characterizing biological tissue. The background signal from tissue autofluorescence and Raman scattering from fused silica in the fiber is also less in this range.

For practical biomedical and diagnostic applications, to identify a possible disease or pathology, it is desirable that Raman spectroscopy can be applied to in vivo tissue, and useful spectra generated as quickly as possible with the maximum amount of information.

Characteristically, precancer or early cancer typically initiates in shallow tissue layers, and when testing for precancerous or early cancer with high accuracy, it desirable to limit the captured Raman photons to those from the surface or epithelial tissue, for example at depths less than 500 μm.

In some circumstances, a tissue autofluorescence background signal as discussed above can originate from relatively deep tissue. This can be a problem when it is particularly desired to perform Raman tissue measurements for surface or epithelial tissue, where the AF signal can interfere with the relatively weak Raman signal from the surface tissue. Where tissue has a number of layers, Raman photons may originate in layers which are not of interest, thus interfering with the spectrum from the layer under investigation. It is desirable for a spectroscope to reduce or exclude as far as possible autofluorescence photons and/or Raman photons from other tissue layers when testing for precancer.

### SUMMARY OF THE INVENTION

According to a first aspect of the invention, we provide a probe head for a diagnostic instrument, the probe head comprising a transmission optical fiber, a plurality of collection optical fibers, and a lens to transmit light from the transmission optical fiber to a test site, wherein the ends of the collection optical fibers are bevelled.

Each bevelled end of a collection fiber may comprise an end face which is at an angle relative to a plane perpendicular to a longitudinal axis of the collection optical fiber.

The end face may be angled in a direction away from the transmission optical fiber. Alternatively, the end face may be angled in a direction towards the transmission optical fiber.

The angle of the end face may be in the range 0° to 25°.

The angle of the end face may be in the range 0° to 20°.

The angle of the end face may be in the range 10° to 15°.

The lens may be spaced from an end face of the transmission optical fiber.

The distance from the lens to an end face of the transmission optical fiber may be less than 1000 μm.

The collection optical fibers may be arranged in a ring around the transmission optical fiber.

The lens may comprise one of; a ball lens, a convex lens, a biconvex lens, an axicon lens, a gradient index lens or a lens system consisting of several lenses.

The probe head may further comprise a narrowband filter associated with the transmission optical fiber.

The narrowband filter may comprise a filter disposed on one of; the distal end of the transmission optical fiber, the lens, and a plate located between the transmission optical fiber and the lens.

The probe head may further comprise a long-pass filter associated with the collection optical fibers

The long-pass filter may be disposed on one of; the distal ends of the collection optical fibers, the lens, and a plate located between the collection optical fibers and the lens.

According to a second aspect of the invention there is provided a diagnostic instrument comprising a monochromatic light source, and a probe head according to the first aspect of the invention, such that light from the monochromatic light source is transmitted through the transmission optical fiber, and spectral analysis apparatus to receive light from the collection optical fibers, the spectral analysis apparatus comprising a grating element, the spectral analysis apparatus further comprising a light-sensing apparatus, wherein the grating element is arranged to diffract light onto an area of the light-sensing apparatus.

The diagnostic instrument may comprise an instrument head to receive the probe head, wherein the probe head extends beyond an end of the instrument head to permit the lens to be placed in direct contact with tissue during measurements.

The grating element may comprise one of a transmission grating and a reflection grating.

The diagnostic instrument may further comprise a processing apparatus, the processing apparatus being operable to receive data from the light-sensing apparatus and generate an output.

The light-sensing apparatus may comprise a sensor array and the data may comprise pixel values

The data may be checked for saturation and rejected if saturation is found.

Generating a spectrum may comprise binning corresponding pixels.

Generating a spectrum may comprise subtracting a background signal from the received data.

Generating a spectrum may comprise smoothing the received data.

Generating a spectrum may comprise fitting a polynomial curve to the smoothed received data and subtracting the fitted curve from the smoothed received data.

The diagnostic instrument may be operable to check the spectra for contamination and if the spectra are valid, classify the spectra as corresponding to healthy or abnormal tissue and generate an output accordingly.

According to a third aspect of the invention there is provided a method of performing a biopsy, comprising using a diagnostic instrument according to the second aspect of the invention, testing a tissue location, receiving a classification of the spectrum as corresponding to healthy or abnormal tissue, and if the tissue is abnormal, taking a sample.

### BRIEF DESCRIPTION OF THE DRAWINGS

An embodiment of the invention is described by way of example only with reference to the accompanying drawings, wherein;

FIG. 1A is a diagrammatic illustration of a diagnostic system embodying the present invention,

FIG. 1B shows a diagrammatic illustration of an instrument head of the endoscope of FIG. 1A,

FIG. 1C is a diagrammatic illustration of the spectrograph of FIG. 1A,

FIG. 2A is a diagrammatic illustration of a probe head embodying the present invention for use with the instrument head of FIG. 1B,

FIG. 2B is a diagrammatic illustration of a further probe head embodying the present invention for use with the instrument head of FIG. 1B,

FIG. 2C is a diagrammatic illustration of a further probe head embodying the present invention for use with the instrument head of FIG. 1B,

FIG. 2D is a side view of a ball lens for use with the probe head of FIG. 2C,

FIG. 2E is a diagrammatic illustration of a further probe head embodying the present invention for use with the instrument head of FIG. 1B,

FIG. 2F is a diagrammatic illustration of a further probe head embodying the present invention for use with the instrument head of FIG. 1B,

FIG. 2G is a perspective view of a plate for use in the probe heads of FIGS. 2E and 2F,

FIG. 2H is a diagrammatic illustration of a further probe head embodying the present invention incorporating a half-ball lens for use with the instrument head of FIG. 1B,

FIG. 2I is a diagrammatic illustration of a further probe head embodying the present invention incorporating a half-ball lens for use with the instrument head of FIG. 1B,

FIG. 2J, is a diagrammatic illustration of a further probe head embodying the present invention incorporating a biconvex lens for use with the instrument head of FIG. 1B,

FIG. 2K is a diagrammatic illustration of a further probe head embodying the present invention incorporating a biconvex lens for use with the instrument head of FIG. 1B,

FIG. 2l is a diagrammatic illustration of a further probe head embodying the present invention incorporating a biconvex lens for use with the instrument head of FIG. 1B,

FIG. 3 is a diagrammatic illustration of a known probe head for use with the instrument head of FIG. 1B,

FIG. 4 is a flow diagram showing a method of operating the system of FIG. 1A,

FIG. 5 is a flow diagram, showing part of the method of FIG. 4 in more detail,

FIG. 6 is a spectrum showing Raman scattering within the probe,

FIG. 7A is a graph showing the simulated and measured efficacy of the probe head of an instrument including the probe head of FIG. 3,

FIG. 7B is a plot showing the origin of Raman photons in a 2-layer tissue model,

FIG. 7C is a graph showing the depth of origin of Raman photons in a 2-layer tissue model,

FIG. 8A is a comparison of raw spectra obtained with the probe heads of FIG. 2A and FIG. 3,

FIG. 8B is a comparison of processed spectra obtained with the probe heads of FIG. 2A and FIG. 3,

FIG. 9 is a graph showing the ratio of Raman photons to autofluorescence photons captured at different anatomical sites with the probe heads of FIG. 2A and FIG. 3,

FIG. 10A shows spectra from normal and abnormal tissue captured using the probe head of FIG. 2,

FIG. 10B shows the principal component loadings for the normal and abnormal spectra of FIG. 9A, and

FIG. 10C is a plot of the first and second principal component scores for differentiation between normal and abnormal spectra.

### DETAILED DESCRIPTION OF THE PREFERRED EMBODIMENTS

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

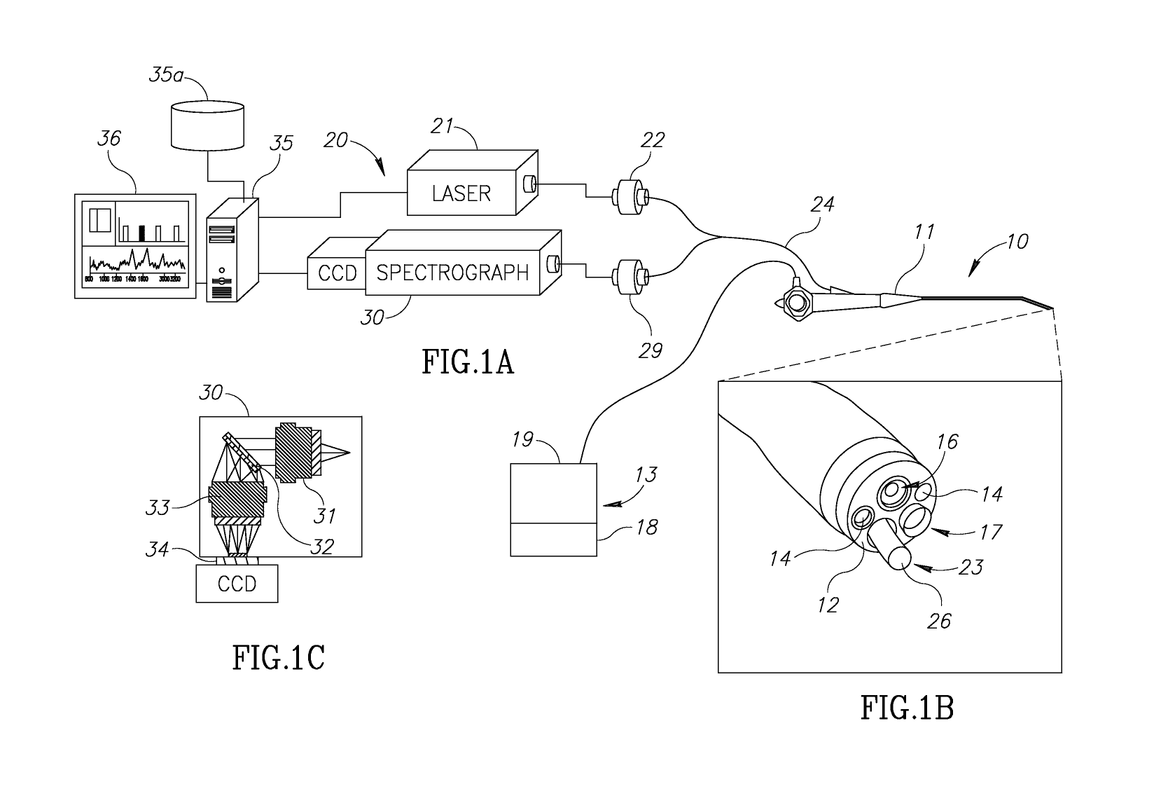

Referring now to FIG. 1A, a diagnostic instrument comprising an endoscope system generally embodying the invention is shown at 10. The endoscope itself is shown at 11 and an instrument head 12 of the endoscope 11 is generally illustrated in FIG. 1A. To provide for guidance and visual viewing of the area being tested, the endoscope 11 is provided with a suitable video system in general shown at 13. Light from a xenon light source 14 is transmitted to illumination windows 15 in the end of the endoscope 12. CCDs 16 and 17, responsive to white light reflection imaging, narrowband imaging or autofluorescence imaging, receive the reflected light and transmit data to a video processor generally shown at 18. The video information is displayed on a monitor diagrammatically illustrated at 19. The video system 13 allows for visual inspection of the tested tissues and for guidance of the endoscope to a desired position.

The Raman spectroscopy apparatus is generally shown at 20. A monochromatic laser source is shown at 21, in the present example a 300 mW diode laser with an output wavelength of about 785 nm. Light from the laser diode 21 is passed through a proximal band-pass filter 22, comprising a narrowband-pass filter being centred at 785 nm with a full width half max of ±2.5 nm. The light is passed through a coupling into a transmission optical fiber provided as part of a fiber bundle 24 leading to a probe head described in more detail below. Scattered light from a tissue test site, returned by a plurality of collection optical fibres as discussed below, is passed through a proximal inline collection long-pass filter 29 which has a cutoff at ˜800 nm. As illustrated in FIG. 1C, the scattered returned light from the collection optical fibers is fed into the spectrograph 30, collected by lens 31 and passed through onto a grating 32, comprising a transmissive diffraction grating. The diffracted light from grating 32 is focussed by lens 33 onto a light-sensing array 34, in the present example a charge-couple device (‘CCD’) comprising a 1340×400 pixel array with a pixel spacing of 20×20 microns.

In the present example, data from the CCD 34 is performed in software on a processing apparatus comprising a personal computer 35, which interfaces with and controls the CCD 34 and laser 21, performs binning and read out of the CCD 34 and carries out the analysis of the spectra. It will be apparent that any other processing apparatus with any suitable combination of general purpose or dedicated hardware and software may be used. A database of spectra used in outlier detection and diagnostic steps is shown diagrammatically at 35a. It will be clear that the database may be stored on the computer 35 or remotely and accessed as needed. The data is processed in real time, in the present example in less than 0.1s. As the spectra are acquired with an integration time of ˜0.5s, the system is suitable for use in real time.

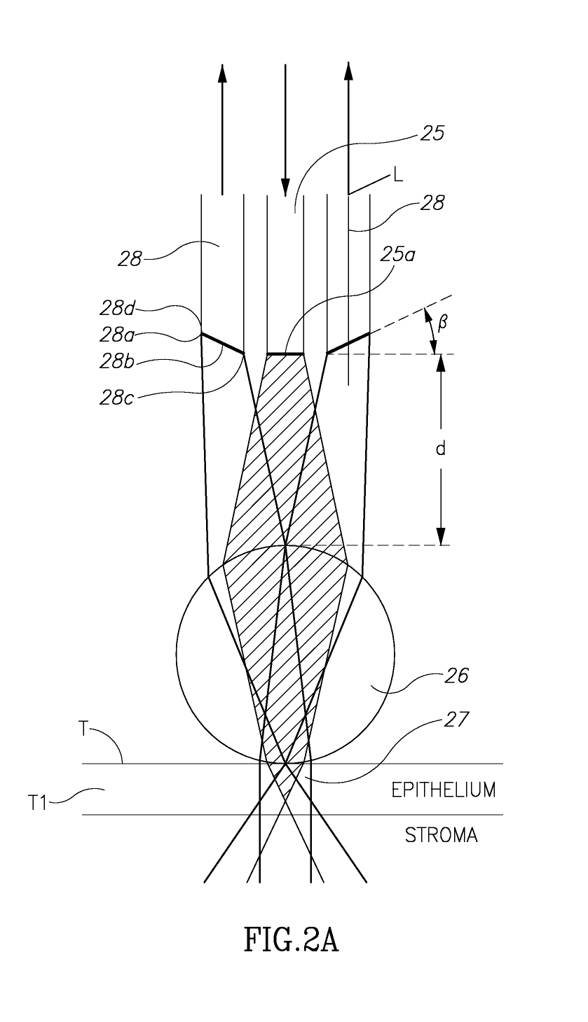

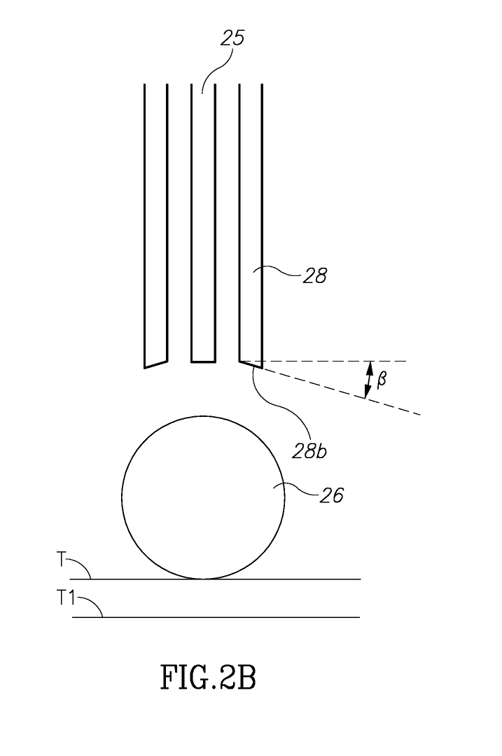

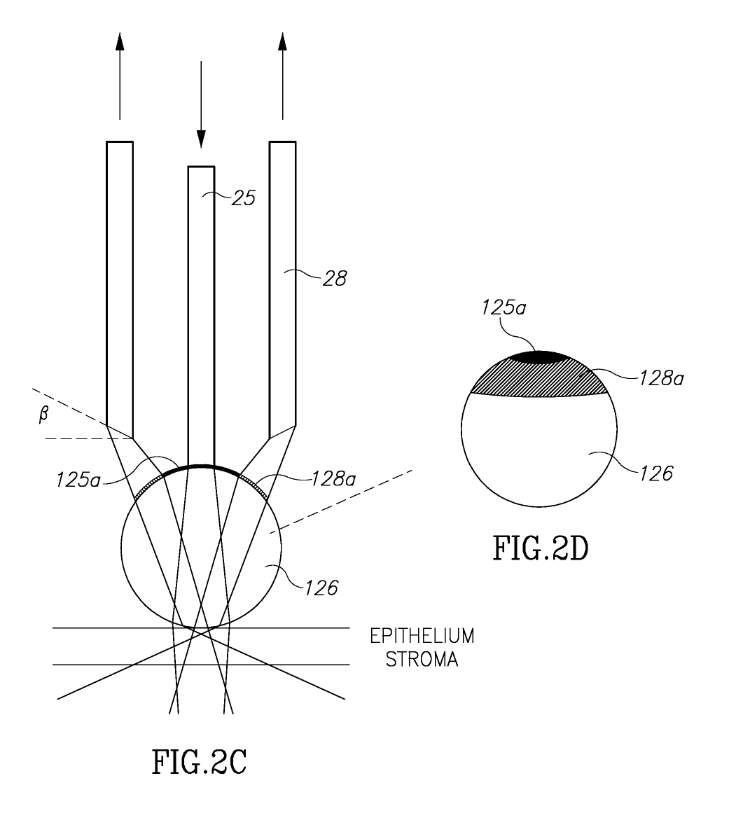

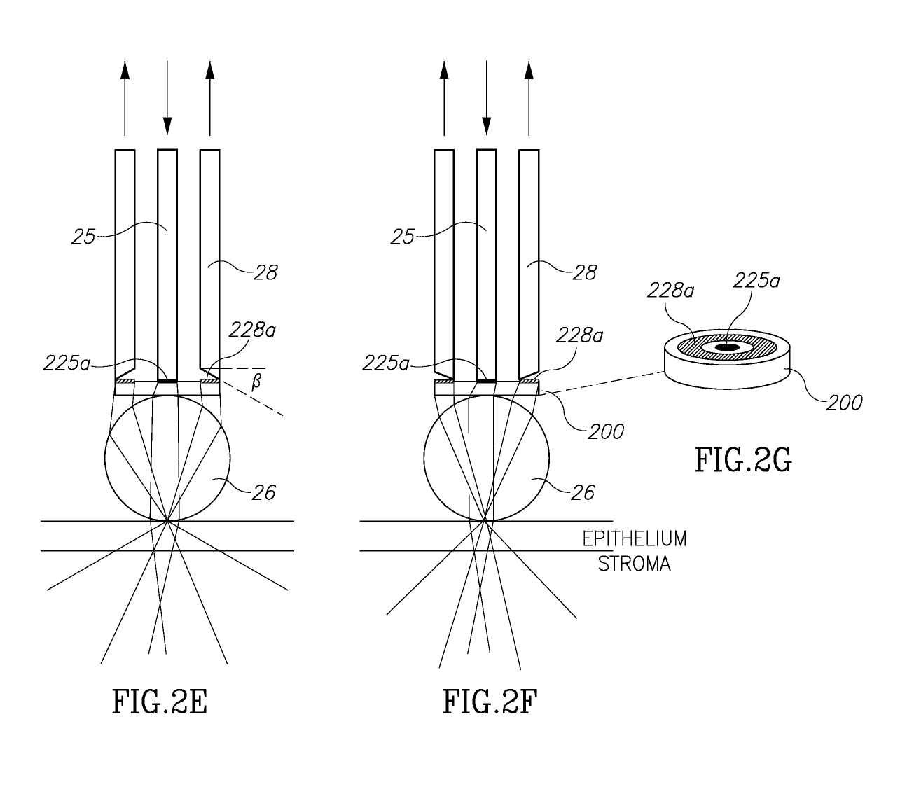

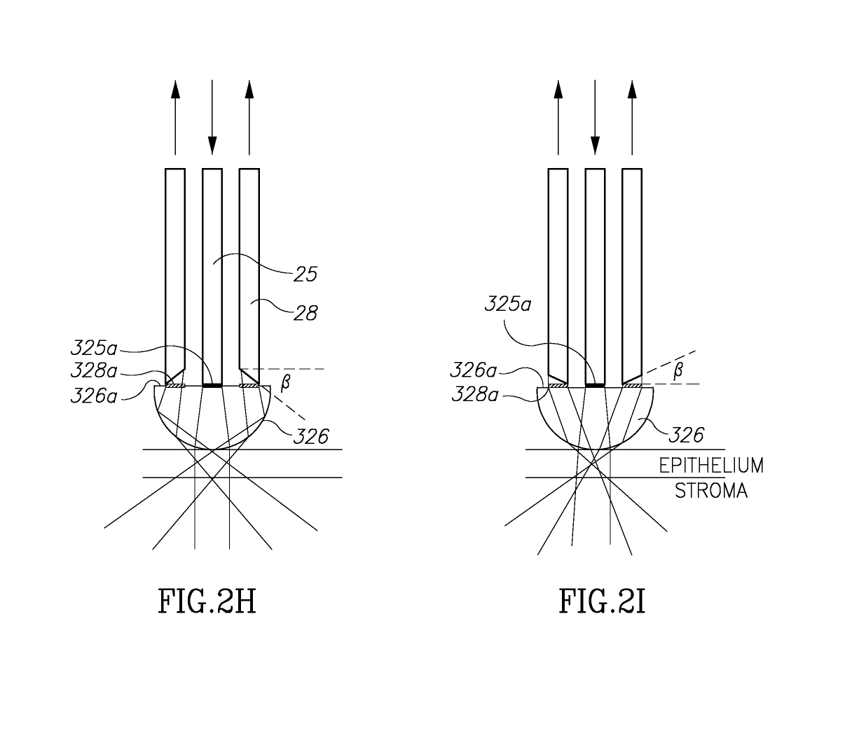

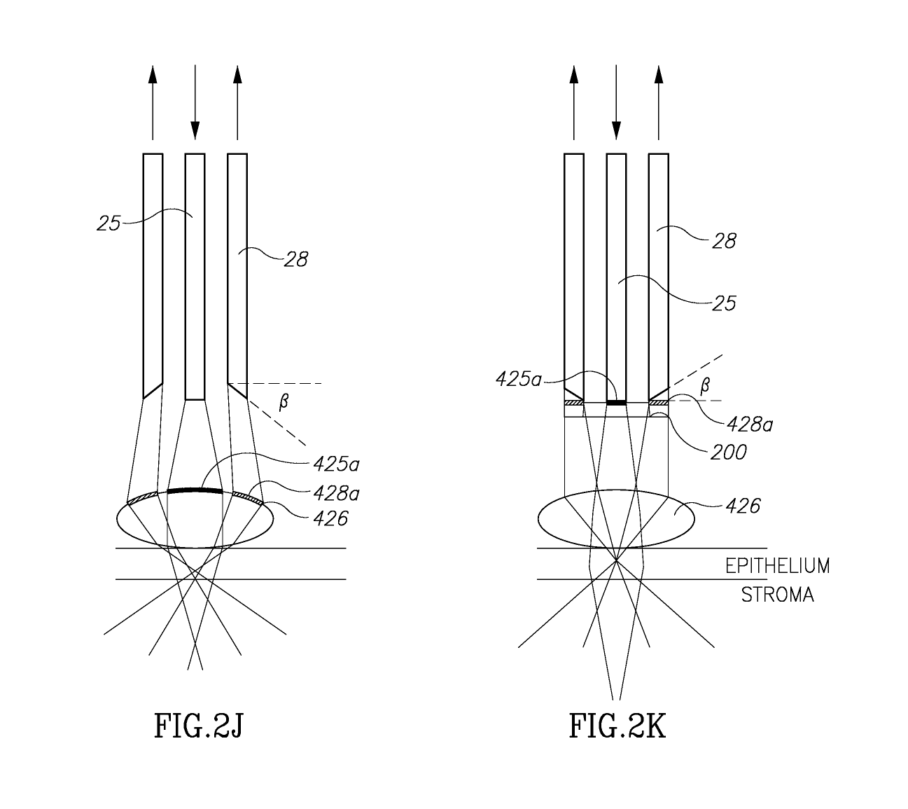

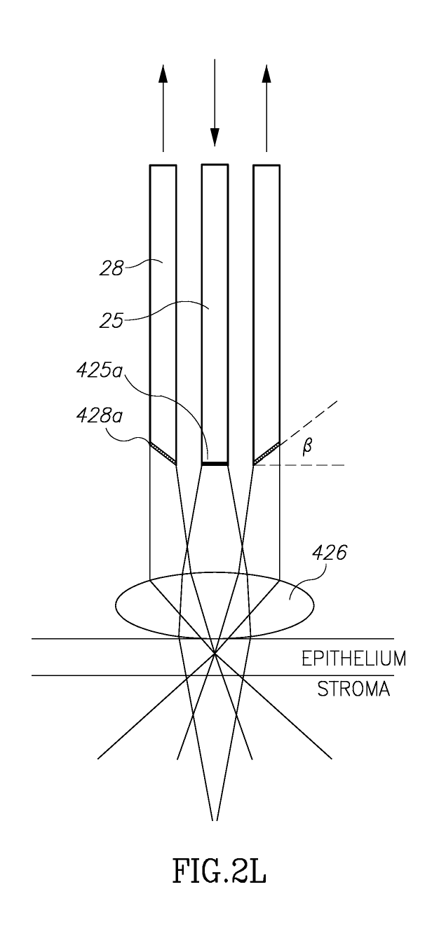

A probe head or ‘confocal probe’ is shown at 23 in FIG. 1B and shown in more detail in FIGS. 2A and 2B. The probe head 23 extends beyond an end of the instrument 12 to enable a lens at the end of the probe head 23 to be placed in contact with tissue to be tested to allow a measurement to be made. A transmission optical fiber 25 is provided as part of the fiber bundle 24 to transmit light from the laser diode 21 to the tissue test site T. The transmission optical fiber has a diameter of 200 μm and a numerical aperture (‘NA’) Of 0.22. A distal band-pass filter 25a is located at the instrument head end of the transmission optical fiber 25, in the present example comprising a coating deposited on the end of the fiber 25. The distal band-pass filter 25a has the same band-pass characteristics as the proximal band-pass filter 22. Light transmitted by the excitation fiber 25 enters a ball lens 26 at the end of the endoscope 11, spaced by a distance d from the end of the transmission optical fiber 25. As illustrated in FIG. 1B, transmitted light from the transmission optical fiber 25 is focussed by the ball lens 26. Where the ball lens is in contact with the tissue to be tested, as shown here at 27, the transmitted light from the transmission fiber 25 at least in part undergoes Raman scattering within the tissue T, confined to a large part within an upper layer T1 of the tissue. The scattered light is again focussed by the ball lens 26 and received in a plurality of bevelled collection optical fibers 28, also provided as part of the fiber bundle 24 to selectively capture those Raman photons from T1. In the present example nine collection optical fibers are provided, each with a diameter of 200 μm and an NA of 0.22. The collection optical fibres 28 may be arranged in any suitable configuration, for example in a ring or circular arrangement surrounding the transmission optical fiber 25, although the fibers may be arranged in any other pattern.

In the present example the ball lens 26 comprises a sapphire ball lens with a diameter of about 1.0 mm and a refractive index n=1.77. The ball lens may alternatively be made of any other material depending on the required refractive index and lens characteristics, such as UV-fused silica (refractive index n=1.46), boron-crown glass (n=1.51), dense flint glass (n=1.63), lanthanum-flint glass (n=1.83) or otherwise. The diameter may be less than 1 mm, for example 500 μm or less, or may be greater than 1 mm. The lens may be provided without a coating, or may have a near-IR anti-reflective coating to reduce specular reflection within the fiber probe. This will reduce the number of backscattered photons within the probe itself, thus reducing undesirable Raman scattering and autofluorescence within the probe, while increasing the tissue Raman signal generation and collection efficiency.

The collection optical fibers 28 are provided with a distal inline long-pass filter 28a at the instrument head end. In a similar manner to the distal band-pass filter 25a, the distal inline long-pass filter 28a is formed as a coating deposited on the end of each collection fiber 28, and has a cut-off at ˜800 nm, thus blocking light from the laser source 21 which has not undergone Raman scattering. The configuration of sapphire ball lens 26, excitation and collection fibers 25, 28, proximal and distal band-pass filters 22, 25a, and distal and proximal long-pass filters 28a, 29 provides a good system for selectively collecting backscattered Raman photons from the tissue T. Although in this example both the distal band-pass filter 25a and distal long-pass filters 28a are shown as coatings provided on the fiber ends, one or both of the filters may be provided on the lens or on a separate substrate as shown in more detail below.

Each collection optical fiber 28 is provided with a bevelled end generally shown at 28b. Each bevelled end is a flat face, with a bevel angle β relative to a plane perpendicular to a longitudinal axis L of the fiber 28.The end faces 28b are arranged such that they are inclined away from the transmission optical fiber 25, i.e. such that a leading edge 28c of each end face 28b is located towards the transmission optical fiber 25, and such that a trailing edge 28d of each end face 28b is located away from the transmission optical fiber 25.

The end faces may alternatively be oriented such that the end faces 28b are directed towards the transmission optical fiber 25 as shown in FIG. 2B, i.e. such that a leading edge 28c of each end face 28b is located away from the transmission optical fiber 25, and such that a trailing edge 28d of each end face 28b is located towards from the transmission optical fiber 25.

In either example, the bevel angle β and spacing d control the light propagation can be chosen in accordance with the specific tissue to be tested and the depth of the layer(s) under investigation, and to exclude Raman photons and/or NIR autofluorescence photons from deeper tissue layers. For example, the probe head may be configured to selectively collect photons from epithelial tissue at depths less than 500 μm, although any appropriate depth range may be selected. Typically β is less than 25°, may be about 20° or even in the range 10-15°. d may be more than 1000 μm, less than 1000 μm, less than 600 or 300 μm, or may even be 0 depending on the tissue and instrument characteristics required. It is envisaged that a probe head would be manufactured with specific characteristics, rather than being adjustable, to allow for a compact package for inclusion in an endoscopic instrument head.

The probe head 23 is sufficiently compact that it can be removed and easily used with conventional instrument heads, such as the instrument head 11 of FIG. 1.

The lens need not be a ball lens. Any other suitable type of lens or lens system may be used, such as a half ball lens, convex lens, biconvex lens, axicon lens or gradient index (‘GRIN’) lens, as examples. Although a single lens is shown here, it will be apparent that a lens system including a plurality of lens, optionally of different types, may be used. By selecting the lens type, bevel angle β and spacing d, the depth of focus and collection volume can be controlled or selected according to the desired function. The combination of bevelled fiber end, lens type and spacing provides an additional degree of freedom compared to simply using a bevelled end fiber or lens alone, thus providing more control of light paths through the probe head and allowing the design of a compact probe, desirable in endoscopic applications.

Alternative configurations of the probe head are shown in FIGS. 2C to 2L.

In FIGS. 2C and 2D, a probe head is shown in which the filters are not provided on the transmission and collection optical fibers 25, 28. Instead, band-pass filter 125a and long-pass filter 128a are provided on a ball lens 126. In this example, band-pass filter 125a comprises a circular element on the surface of the ball lens 126, and the long-pass filter 128a comprises an annular band surrounding the band-pass filter 125a. The configuration of the filters 125a, 128a is selected to conform to the geometry of the fibers 25, 28 and the spacing d, such that the light paths to and from the respective fibers pass through the filters 125a, 128a. In this example, the spacing, geometry and filter arrangement are selected such that there is no overlap both between the cone of light from the transmission optical fiber 28 and the long-pass filter 128a, and between the collection light cone and the band-pass filter 125a.

In FIGS. 2E to 2G, the filters are provided on a plate 200 located between the fibers 25, 28 and the lens 26. Band-pass filter 225a comprises a circular area, while long-pass filter 228a comprises an annular band extending around the band-pass filter 225a. The filters 225a, 228a may be spaced as shown here, or may abut. The plate 200 in this example comprises a glass plate with a thickness of at least 0.1-0.3 mm, such as quartz or sapphire, that preferably is less Raman active in the wavenumber range under investigation (for example 400-3600 cm⁻¹). The filter 228a need not be continuous, but could for example be a plurality of discrete spaced areas, depending on the fiber geometry and spacing between the fibers 25, 28, plate 200 and lens 26. The plate 200 need not be flat.

In FIGS. 2H and 2I, the lens comprises a half-ball lens 326. The half-ball lens may be made of any suitable glass as discussed above. A flat face 326a of the ball lens 326 is directed towards the distal ends of the fibers 25,28, in this example with spacing d=0. A band-pass filter 325a and long-pass filter 328a are formed on the flat face 326a, the arrangement of the filters 325a, 328a depending on the geometry of the fibers, lens and spacing d.

In FIGS. 2J to 2L, a probe head having a biconvex lens 426 is shown, with alternative locations of the band-pass filter 425a and long-pass filter 428a shown. According, in FIG. 2J the filters 425a, 428a are deposited on an upper surface 426a of the lens 426. In FIG. 2K, the filters 425a, 428a are provided on a plate 200 abutting the transmission optical fiber 25, as in FIGS. 2e to 2g. In FIG. 2L, the filters 425a, 428a are deposited on the distal ends of the optical fibers 25, 28 as in FIG. 2A.

The configurations in FIGS. 2A to 2L are not exclusive, and it will be apparent that any combination of lens and filter arrangement can be used. It will be further apparent that the filters need not be provided on the same element; for example, one filter could be provided on a plate and one on a lens surface, or on a fiber end, or any combination.

In general, the band-pass filter in the present example is a narrowband filter centred at 785 nm with a full width at half max of ±2.5 nm. The long-pass filters have a cut-off at 800 nm and high transmission in the range 800-1200 nm. Alternative filters may be used depending on the source wavelength and the range of desired collection wavelengths.

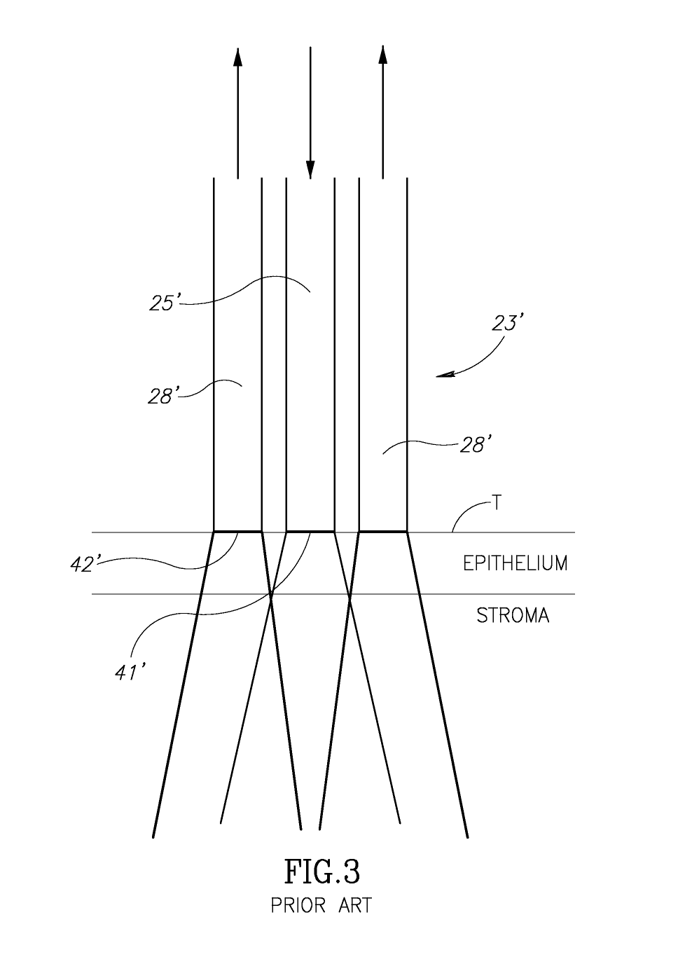

A known probe head, or ‘volume probe’ for use with the instrument head 12 is shown at 23′ in FIG. 3 for comparison purposes. The known probe head 23′ comprises a central transmission optical fiber 25′ surrounded by a bundle of collection optical fibers 28′. As shown in FIG. 3, the ends 41′, 42′ of the optical fibers 24′, 28′ are essentially aligned and, in use, abutted against the tissue test site T′.

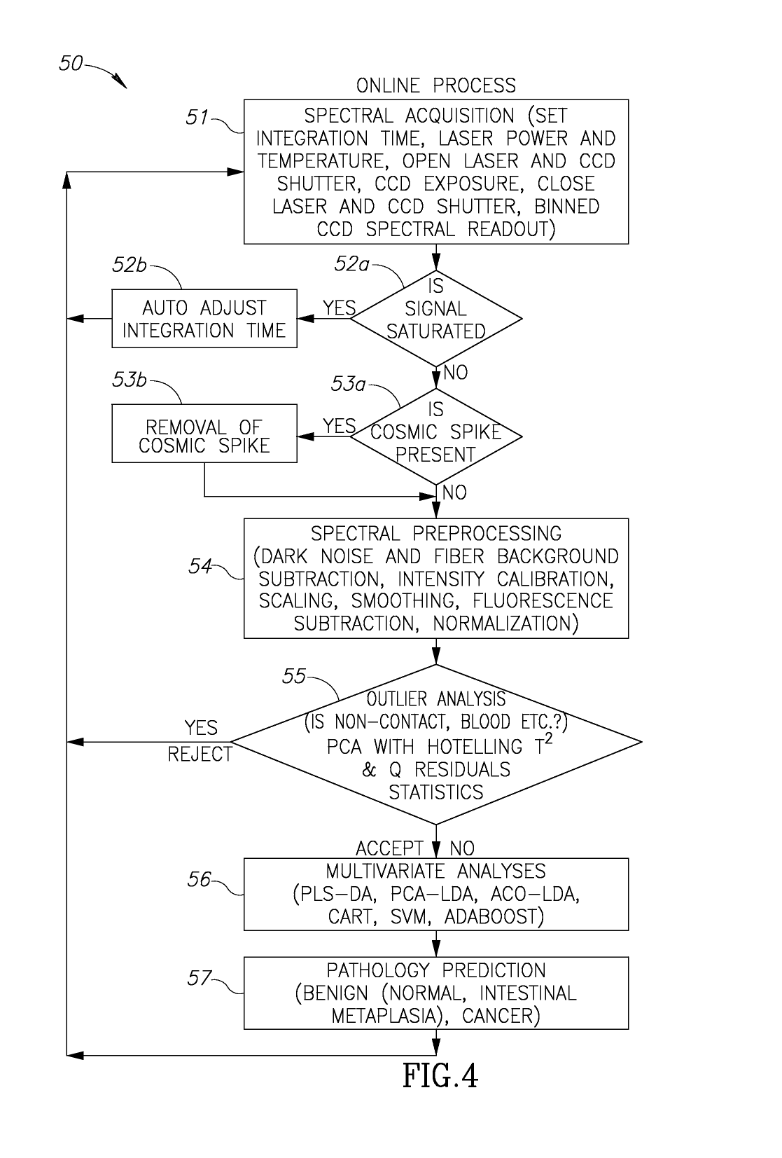

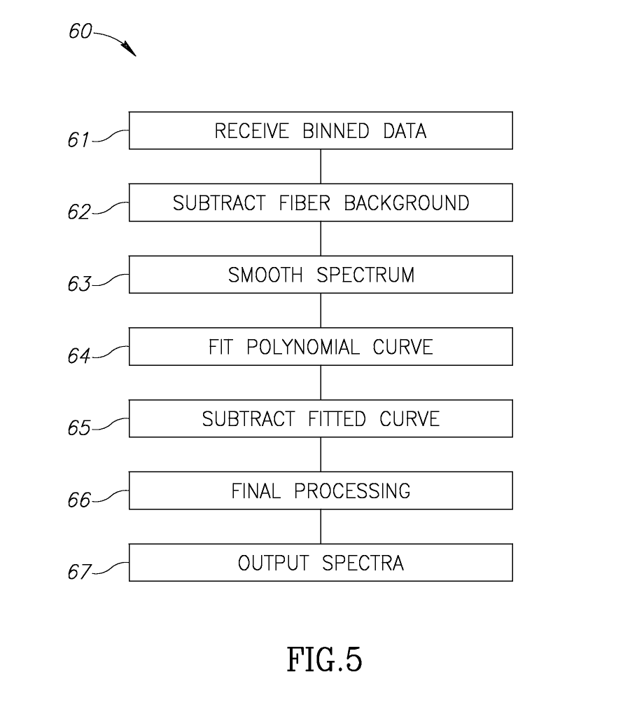

As discussed above, the light scattered from tissue T, T′ is returned to the spectrograph 30 and is captured by the CCD 34, and the Raman spectra extracted. The image data from the CCD 34 is processed in the following manner, with reference to FIGS. 4 and 5. The processing method is illustrated at 50 in FIG. 4. At step 51, the CCD integration time, laser power and temperature are set. Light from the laser is sent to the probe head 23 and reflected light passed to the spectrograph 30, for example by opening one or more shutters. After a set CCD exposure time, the shutter is closed. The pixel values from CCD 34 are binned and read out, to maximise the signal to noise ratio at each wavelength. At step 52a, the data is checked for saturation, i.e. whether any of the pixel values are at a maximum value. If so, then at step 52b the integration time of the CCD 34 is adjusted and a new image acquired with a shorter integration time which is acquired at step 51. At step 53a the data is checked for the characteristics of a spike caused by cosmic rays, and if so the spike is removed at step 53b.

If the signal is not saturated, then at step 54 the spectrum is preprocessed as discussed in more detail below with reference to FIG. 5. At step 55, outlier detection is performed, to check that the spectrum from step 54 corresponds to a valid signal from tissue and not from contaminants. If the spectra are not valid, the spectra are rejected and new images are acquired at step 51.

In the present example, the outlier detection step is performed using principal component analysis (‘PCA’) of the captured spectra compared to a database or library of stored spectra, diagrammatically illustrated at 35a. The library of spectra contains spectra from healthy, abnormal and pre-cancerous tissue. PCA is a known method of analysing a data set by characterising the variability of the data set in terms of a smaller number of variables—the principle components-, their relative weights, and an error term for each group of values corresponding to a particular measurement which is a measure of how well the derived principal components match that measurement. In this case PCA is able to reduce the high dimensionality of the library of stored spectra to a smaller number of variables, typically 2 to 5, which forms a model which can be stored for subsequent use. By using the error term, a captured spectrum can be assessed as a genuine spectrum or an outlier. In the present example, the Hotelling T² and Q-residual statistics are calculated. The Q-residual statistic is an indicator of how good or bad a fit the derived model is to the measured data, while the T² statistic is a measure of how far the measurement is from the mean or centre of the model.

When a new spectrum is captured, PCA is performed on the new spectrum and the Hotelling T² and Q-residual statistics are calculated. Only spectra within the 95% or 99% confidence interval of both the T² and Q-residual statistics of the stored model are accepted. Spectra in the 95% confidence interval for both statistics are stored and if the Hotelling T² and Q-residual statistics for measured spectra lie outside this region, they are rejected as outliers. It will be apparent that the library of spectra is selected such that genuine spectra from abnormal tissue are not rejected.

If the spectra are valid, then at steps 56 and 57 further processing steps may be performed, for example to identify spectral characteristics associated with cancerous or precancerous cells, or with other diseases or disorders. In this example, the library of stored spectra may once again be used, as it contains examples of healthy, precancerous and cancerous tissue and may be used in a suitable manner to classify the captured spectra. Alternatively, separate libraries may be used for each step if appropriate or desirable. An example of a suitable technique is probabilistic partial least squares discriminant analysis (‘PLS-DA’), in particular because the aim is to classify tissue into one of two states, healthy and abnormal or cancerous. At step 57, the pathology associated with the results of step 56 and any other desired processing results can be determined, and may be presented on a suitable display 36 or other output.

The processing step 54 is now discussed in more detail with reference to FIG. 5, the method being illustrated at 60. At step 61 the binned spectra are received and, at step 62, the fiber background is subtracted. This is the spectral component from Raman scattering from fused silica within the optical fiber. The fiber background is stored, or captured prior to the test. This removes that part of the returned signal that does not originate from within the tissue.

At step 63, the spectra are smoothed, by using a suitable averaging window or technique. In the present example, Savitzy-Golay smoothing with a window width of 5 pixels is used, as this is found to improve the signal quality in noisy Raman spectra.

At step 64, a polynomial curve is fitted to each of the smoothed spectra. The choice of the order of the polynomial curve fitted depends on the spectral range and shape of the background signal resulting from tissue autofluorescence. In the present example, a third-order polynomial is fitted in the HW region and a fifth-order polynomial in the FP region.

At step 65, the fitted curve is subtracted from the corresponding smoothed spectrum. This removes the background signal while leaving the characteristic Raman spectral peaks.

At step 66, other processing steps are performed to improve visualisation and presentation of the spectra. The spectra can for example be normalised, so that there is a given area under each line, or combined to give an apparently continuous spectrum by averaging the overlapping region, or otherwise. At step 67 the spectra are output for use in the diagnostic and pathology steps 56, 57 of FIG. 3.

The steps shown in FIG. 5 are not intended to be exclusive, and other or additional processing steps or techniques may be used, such as multiple scatter correction. As a further example, although background subtraction is shown, it is possible that the epithelial background autofluorescence signal could be used in conjunction with the Raman signal for diagnosis.

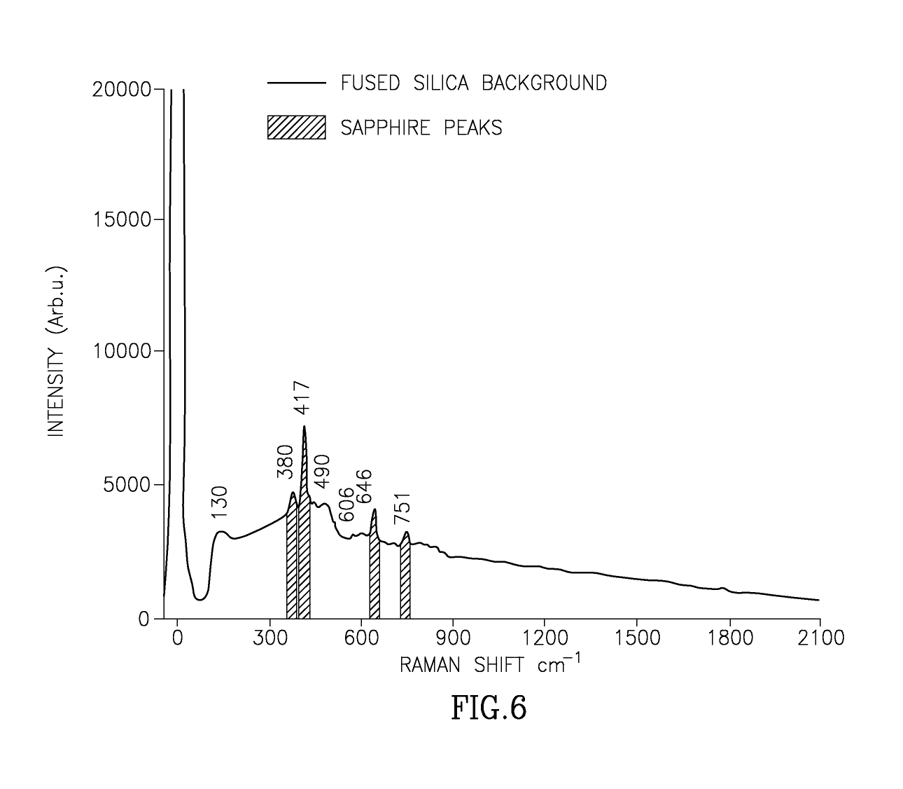

Advantageously, in the processing steps of FIGS. 4 and 5, signals from the lens 26 itself may serve as an internal reference for the laser power and/or system throughput. FIG. 6 shows the background spectrum of a sapphire ball-lens fiber-optic Raman probe used when excited by a 785 nm diode laser. The distinct sapphire (Al₂O₃) Raman peaks originating from the distal ball lens can be found at 417 and 646 cm⁻¹(phonon mode with A_{1g} symmetry), and 380 and 751 cm⁻¹ (E_{g} phonon mode). There are also two dominant Raman components from the fused silica fiber as well as a relatively weak fiber fluorescence background. The sharp “defect peaks” of fused silica denoted as D₁ and D₂ at 490 and 606 cm⁻¹, have been assigned to breathing vibrations of oxygen atoms in four- and three-membered rings, respectively. These characteristic background Raman peaks (shorter than fingerprint region (800-1800 cm⁻¹)) from the fiber-optic Raman probe itself serve as internal reference signals for the tissue Raman measurements.

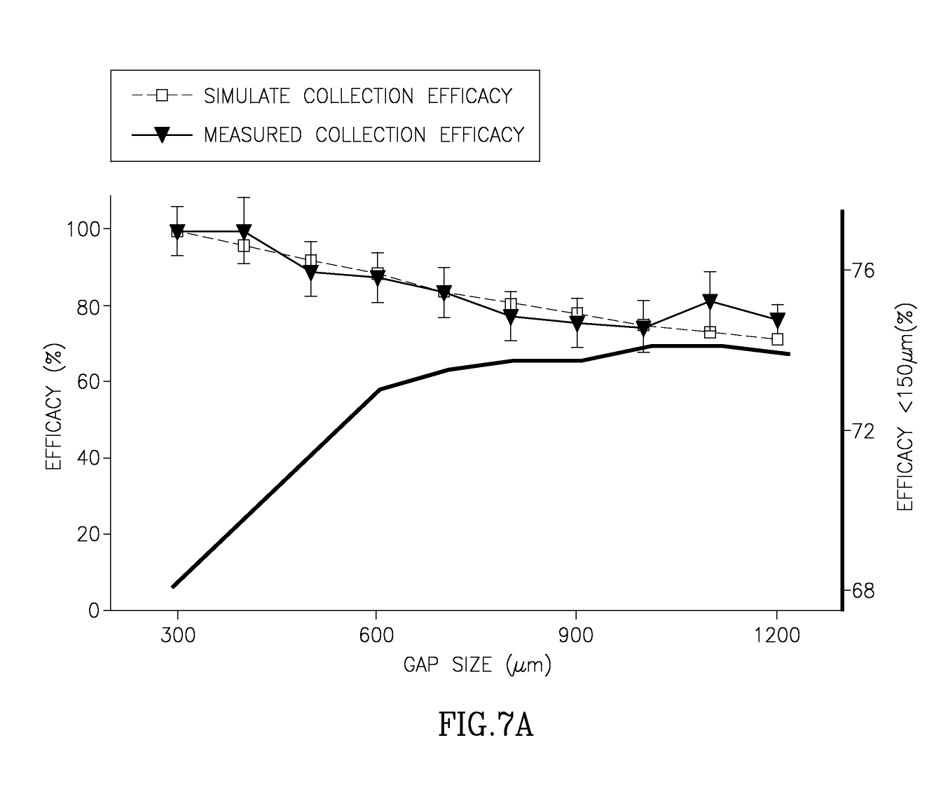

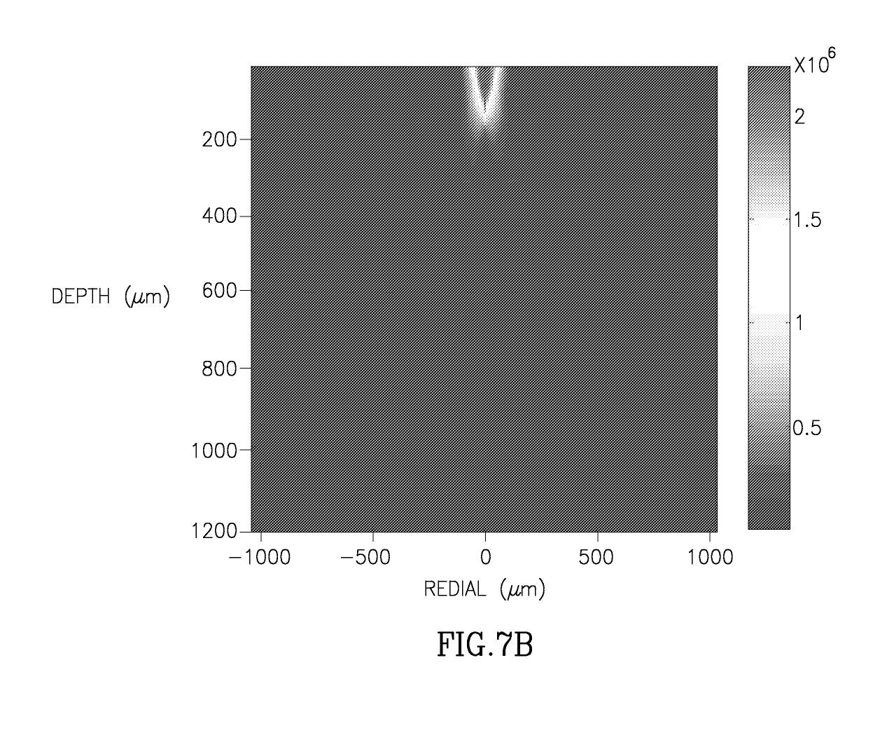

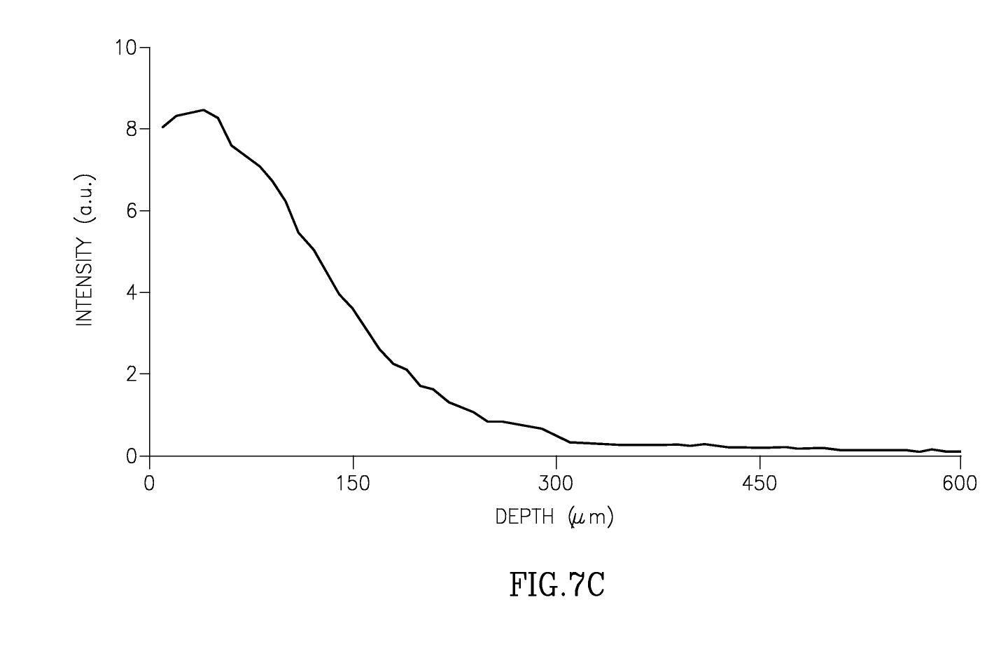

FIGS. 7A to 7C illustrate the expected origin of scattered Raman photons and the collection efficiency of the probe head 23. The upper lines of FIG. 7A show the expected collection efficacy of the probe head 23, estimated using a Monte Carlo simulation, and measured using a Raman probe as described above. The lower line shows the proportion of Raman photons captured as a function of spacing d. FIGS. 7B and 7C show the expected origin of the Raman photons, confined to a tapered volume immediately below the probe head 23 and mostly at a depth of less than 150 μm, i.e. confined to the epithelium. The sampled volume is about 0.01 mm3, compared to about 1 mm3 for the volume probe 26′.

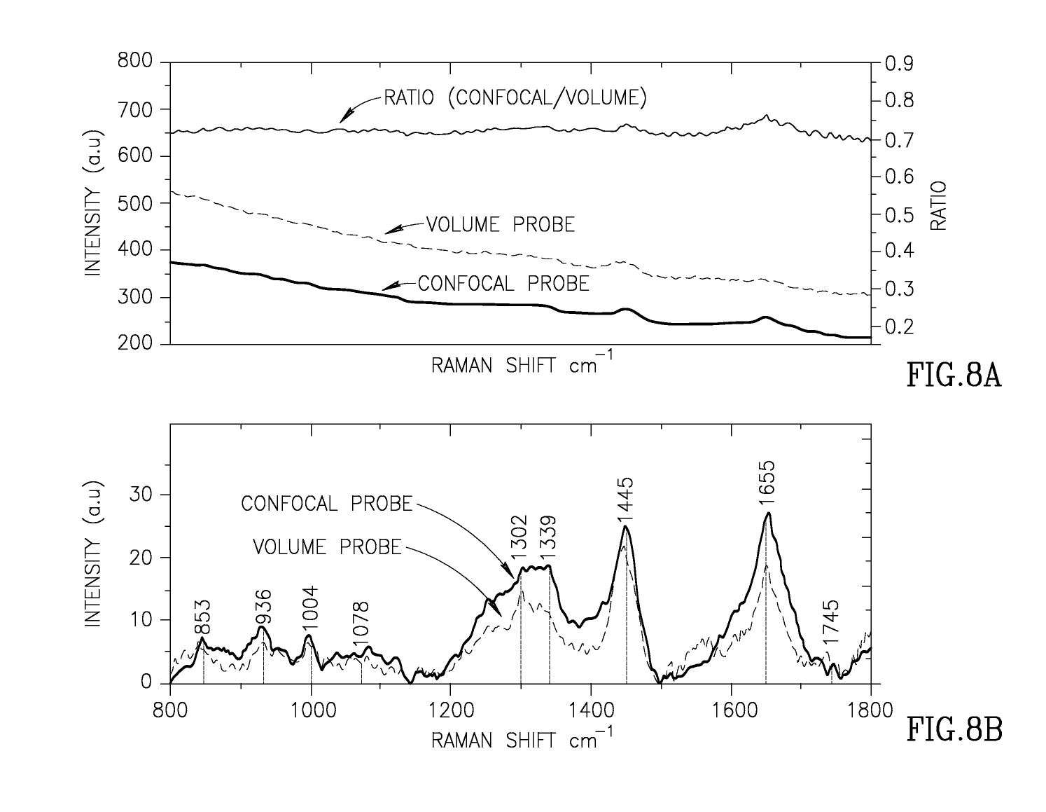

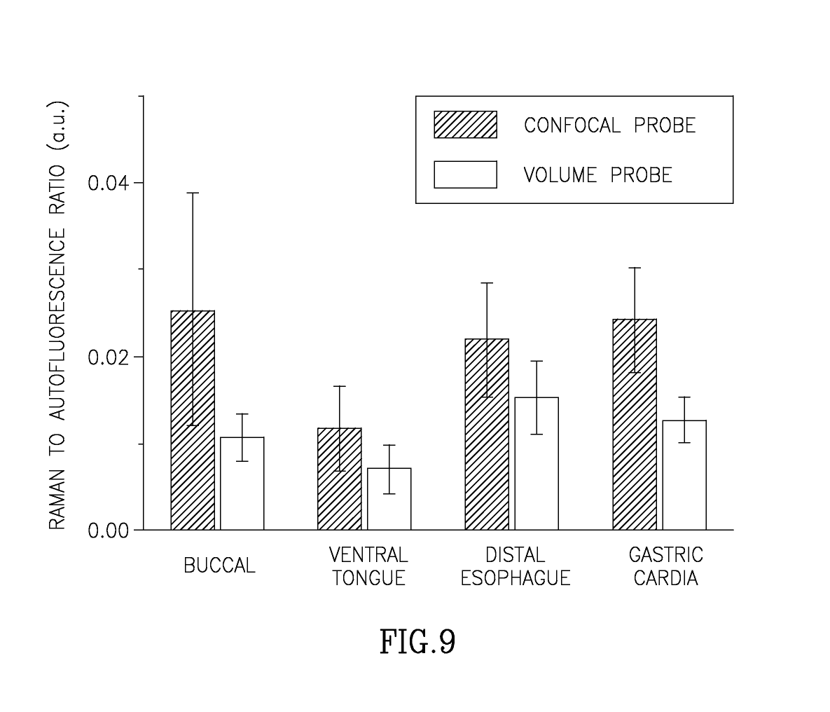

FIGS. 8A and 8B show the results of a comparison of the confocal and volume probes in healthy gastric tissue, with tip powers of 40 mW and 100 mW respectively to obtain comparable irradiances at the surface of the tissue T. In FIG. 8A, the raw spectra (i.e. before background removal at step 64) are compared and the intensity ratio shown. In FIG. 8B, the Raman spectra are shown after autofluorescence background removal. The graphs show that using the confocal probe a better signal-to-noise ratio (‘SNR’) can be obtained compared with the volume probe, and with much less tissue autofluorescence by about 30%, suggesting that autofluorescence within deep tissue is suppressed using the confocal probe head of the present invention. It has further been found that spectra obtained with the confocal probe head of the present invention exhibit much reduced spectral variance compared to a volume probe head. The improved SNR is illustrated further in FIG. 9, where the ratio of Raman photons to AF photons captured at different anatomical sites by a confocal probe and volume probe are compared. The ratios captured using the confocal probe are significantly higher, confirming that the autofluorescence signals from deep tissue are effectively removed using the confocal probe. The sites or organs shown here (buccal, ventral tongue, distal esophagus and gastric cardia) are not exclusive, and it will be apparent that the instrument may be used elsewhere as appropriate, for example for detection of cervical cancer.

Additionally, has been found that as angle β increases, and as the spacing d increases, the number of collected Raman photons fall but the ratio of Raman photons originating in the epithelium rather than the stroma increases. For example, when the angle β is about 20°, the probe head acquires ˜85% of the Raman photons originating in the epithelium and only ˜23% of the photons originating from the stroma. In particular, the probe head was found to have a SNR of about 6 when angle β is about 20° and d is 0.

Accordingly, the probe head disclosed herein is effective at selectively excluding photons from autofluorescence and from other tissue layers. The probe head provides a means of accurately controlling the depth of interrogation, so that probe heads may be used with different tissue types having different epithelia. By capturing more signals from the surface or tissue layer of interest, sensitivity to precancer is increased. The instrument also has a high collection efficiency, making it suitable for real-time endoscopy and diagnosis or tissue classification.

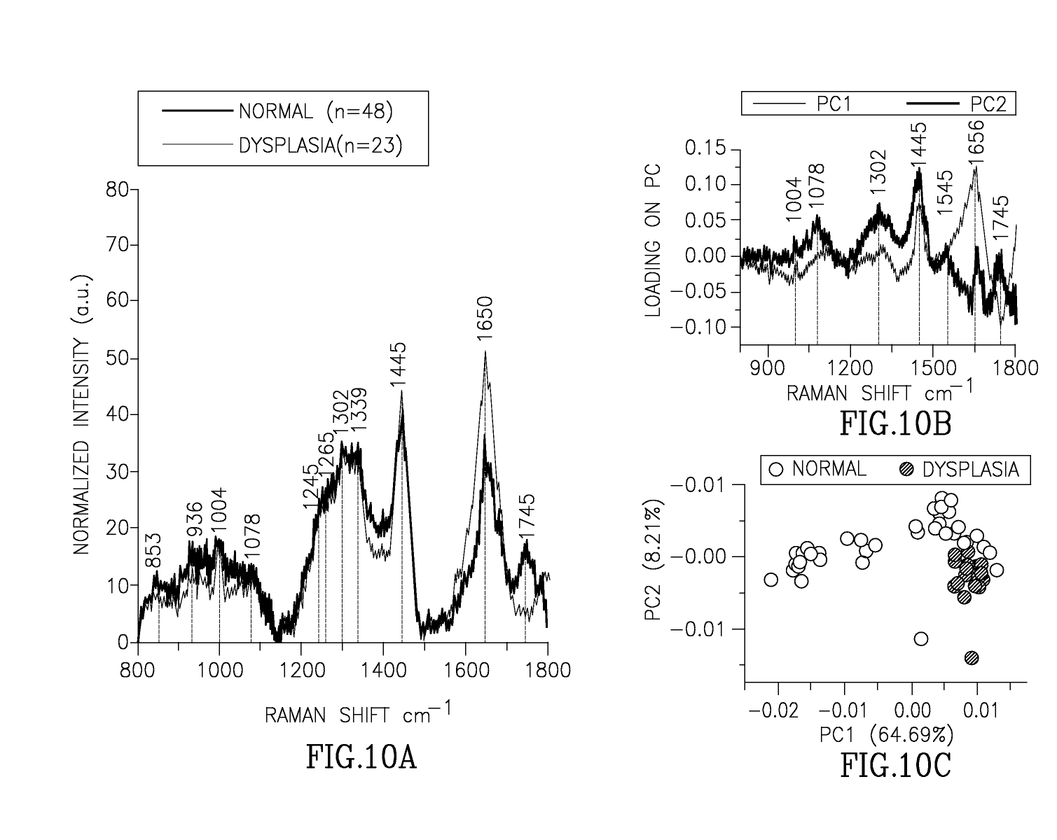

FIGS. 10A to 10C illustrate the use of a diagnostic instrument incorporating a probe head as described above, in accordance with the method of FIGS. 4 and 5. A Raman endoscopy probe including a probe head as described above was used to make in vivo measurements for detection of gastric precancer (dysplasia). FIG. 10A shows the mean in vivo Raman spectra acquired from normal and dysplastic patients. Changes in the spectra, i.e. changes in the peak intensity and bandwidth, can be seen between the normal and abnormal spectra, particularly around 1398, 1655 and 1745 cm⁻¹. FIG. 10B shows the principal component loadings, resolving diagnostically important Raman peaks at 1004, 1265, 1302, 1445, 1665 and 1745 cm⁻¹. As can be seen in FIG. 9C, two-component principal component analysis with spectral variance captured can be used to provide diagnosis of dysplasia, in this example with an accuracy of 85.92%

Although the instrument described herein is an endoscope with visualisation or guidance means, it will be apparent that the invention may be implemented in any other instrument or suitable apparatus, such as a gastroscope, colonoscope, cystoscope, bronchoscope, colposcope or laparoscope etc., for diagnosis or testing for any other suitable condition from those described herein.

The instrument described herein may also be suitable for performing biopsies, in particular for conditions where random samples may yield a large number of negative samples and be time-consuming and distressing, for example in Barrett' esophagus. The instrument may be used to test a potential biopsy site, and the instrument is operated as described above to classify the tested tissue as normal or abnormal. If the received classification shows the tissue is abnormal, a sample can be taken from the site, either immediately, using an attachment on the same instrument, or subsequently.

Although the probe head described herein is intended for use in Raman spectroscopy, it will be apparent that the probe head may be used in any other suitable technique, such as fluorescent or reflectance spectroscopy.

The probe head, diagnostic instrument and method described herein may be suitable for use with the Raman spectroscopy apparatus and methods described in our co-pending applications nos. GB1302886.5 filed on 19 Feb. 2013 and PCT/SG2013/000273 filed on 2 Jul. 2013, the contents of which are included in their entirety by reference.

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

Meanings of technical and scientific terms used herein are to be commonly understood as by one of ordinary skill in the art to which the invention belong, unless otherwise defined.

1. A probe head for a diagnostic instrument, the probe head comprising;
a transmission optical fiber having an end face configured to output light;
a plurality of collection optical fibers; and
a lens spaced a distance d apart from the end face of the transmission optical fiber and configured to transmit light from the transmission optical fiber into epithelial tissue and stroma tissue at a test site,
wherein the ends of the collection optical fibers are bevelled such that each bevelled end of a collection optical fiber comprises an end face disposed at an angle β relative to a plane perpendicular to a longitudinal axis of the collection optical fiber, and
wherein the distance d and angle β are selected such that the plurality of collection optical fibers selectively collect Raman photons from epithelial tissue layers while excluding photons from other tissue layers and while excluding autofluorescence photons.

2. The probe head according to claim 1 wherein number of collected Raman photons originating in epithelial tissue layers rather than stroma tissue layers increases as the angle β increases and the spacing d increases.

3. The probe head according to claim 1 wherein the end face of each of the plurality of collection optical fibers is angled in one of a direction away from the transmission optical fiber and a direction towards the transmission optical fiber.

4. (canceled)

5. The probe head according to claim 1 wherein the angle of the end face is in the range 0° to 25°.

6. (canceled)

7. (canceled)

8. (canceled)

9. The probe head according to claim 1 wherein the distance from the lens to an end face of the transmission optical fiber is less than 1000 μm.

10. The probe head according claim 1 wherein the collection optical fibers are arranged in a ring around the transmission optical fiber.

11. The probe head to according claim 1 wherein the lens comprises one of a ball lens, a convex lens, a biconvex lens, an axicon lens and a gradient index lens.

12. The probe head according to claim 1, further comprising a narrowband filter associated with the transmission optical fiber.

13. The probe head according to claim 12 wherein the narrowband filter comprises a filter disposed on one of the distal end of the transmission optical fiber, the lens, and a plate located between the transmission optical fiber and the lens.

14. The probe head according to claim 1, further comprising a long-pass filter associated with the collection optical fibers.

15. The probe head according to claim 14 wherein the long-pass filter is disposed on one of the distal ends of the collection optical fibers, the lens, and a plate located between the collection optical fibers and the lens.

16. A diagnostic instrument comprising;
a monochromatic light source;
a probe head comprising:
a transmission optical fiber having an end face configured to output light received from the monochromatic light source;
a plurality of collection optical fibers; and
a lens spaced a distance d apart from the end face of the transmission optical fiber and configured to transmit light from the transmission optical fiber into epithelial tissue and stroma tissue at a test site,
wherein the ends of the collection optical fibers are bevelled such that each bevelled end of a collection optical fiber comprises an end face disposed at an angle β relative to a plane perpendicular to a longitudinal axis of the collection optical fiber, and
wherein the distance d and angle β are selected such that the plurality of collection optical fibers selectively collect Raman photons from epithelial tissue layers while excluding photons from other tissue layers and while excluding autofluorescence photons; and
such that light from the monochromatic
and a spectral analysis apparatus configured to receive light from the collection optical fibers,
wherein the spectral analysis apparatus comprises
a grating element, and
a light-sensing apparatus, wherein the grating element is arranged to diffract light onto an area of the light-sensing apparatus.

17. The diagnostic instrument according to claim 16 further comprising an instrument head to receive the probe head, wherein the probe head extends beyond an end of the instrument head to permit the lens to be placed in direct contact with tissue.

18. The diagnostic instrument according to claim 16 wherein the grating element comprises one of a transmission grating and a reflection grating.

19. The diagnostic instrument according claim 16 further comprising a processing apparatus, the processing apparatus being operable to receive data from the light-sensing apparatus and generate an output comprising a spectrum.

20. (canceled)

21. The diagnostic instrument according to claim 19 wherein the processing apparatus is configured to check the received data for saturation and reject the received data if saturation is found.

22. The diagnostic instrument according to claim 19 wherein the processing apparatus is configured to generate a spectrum by way of binning corresponding pixels of the received data, subtracting a background signal from the received data, and smoothing the received data from which the background signal was subtracted.

23. (canceled)

24. (canceled)

25. The diagnostic instrument according to claim 22 wherein processing apparatus is further configured to fit a polynomial curve to the smoothed data and subtract the fitted curve from the smoothed data.

26. The diagnostic instrument according to 16, wherein the processing apparatus is configured to check the spectrum for contamination and determine if the spectrum is valid, and in the event that the spectrum is valid classify the valid spectrum as corresponding to healthy or abnormal tissue.

27. (canceled)
